(19) United States
(12) Patent Application Publication
(10) Pub. No.: US 2025/0003950 A1
(43) Pub. Date: Jan. 2, 2025

(54) SYSTEMS AND METHODS FOR PREDICTING DISEASES

(71) Applicant: VOLATILE ANALYSIS CORPORATION, Grant, AL (US)

(72) Inventor: Katherine Bazemore, Grant, AL (US)

(21) Appl. No.: 18/736,143

(22) Filed: Jun. 6, 2024

Related U.S. Application Data
(63) Continuation of application No. 16/393,598, filed on Apr. 24, 2019, now abandoned.
Application No. 16/393,598 is a continuation-in-part of application No. 15/961,787, filed on Apr. 24, 2018, now abandoned.
(60) Provisional application No. 62/489,062, filed on Apr. 24, 2017.

(51) Int. Cl. G01N 33/497; A61B 5/08; A61B 5/097; A61B 10/00; G01N 1/22; G01N 1/40; G01N 33/68; G06N 3/02; G06N 20/00

(52) U.S. Cl. CPC G01N 33/497; A61B 5/082; G01N 1/4044; G01N 1/405; G06N 20/00; A61B 5/097; A61B 10/0051; A61B 2010/0087; G01N 2001/2244; G01N 33/6893; G01N 2800/52; G06N 3/02

(57) ABSTRACT

A system for predicting diseases may accumulate information about the volatile, semi-volatile, and non-volatile organic compounds in breath/saliva. Such information may be analyzed over time to identify early disease indications, using non-invasive data collection via breath and alert patients directly for follow-up with a health professional.

### CROSS REFERENCE TO RELATED APPLICATIONS

This application is a continuation of and claims priority to U.S. application Ser. No. 16/393,598, entitled “Systems and Methods for Predicting Diseases” and filed on Apr. 24, 2019, which is incorporated herein by reference. U.S. application Ser. No. 16/393,598 is a continuation-in-part of U.S. patent application Ser. No. 15/961,787, entitled “Predictive Disease Breath Database Systems and Methods” and filed on Apr. 24, 2018, which is incorporated herein by reference. U.S. patent application Ser. No. 15/961,787 claims priority to U.S. Provisional Application No. 62/489,062, entitled “Automated Disease Identification Platform” and filed on Apr. 24, 2017, which is incorporated herein by reference.

### RELATED ART

Various techniques for detecting disease have been developed and are instrumental in healthcare. Early detection is important and even sometimes critical in successful treatment for many types of diseases, but such early detection can be difficult. In addition, due to inherent difficulties in detecting many types of diseases, patients are sometimes given incorrect or inadequate diagnosis, which can lead to complications or problems in treatment. Moreover, improved techniques for detecting disease are generally desired.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 shows exemplary inputs and outputs of a system for predicting diseases. The input data may be accumulated using techniques described by U.S. Pat. No. 9,480,461 or other known techniques. Compounds [c] are plotted by concentration, at multiple time snapshots [t], for multiple patients [p]. The output data may be generated by doctors making a diagnosis of some number of disease conditions, each of which is associated with a patient [p] at time [t]. These data are suitable for the parameter/result format used for supervised machine learning techniques.

FIG. 2 shows an exemplary cycle for disease prediction. The prediction cycle involves collecting breath profiles on a regular basis, and escalating to actual medical diagnoses on a less frequent basis. The profiles and diagnoses (both positive and negative) are collected in a database or other form of memory, and are used as input and outputs for model building. The model is applied each time a breath profile is collected, and this is used to make a prediction, which may be made available to the consumer. These predictions may be continuously improved by the cycle actively feeding new data into the system, allowing the models to be refined.

FIG. 3 shows an exemplary process for capturing breath profile. Capturing of a breath profile (an input) can be done by various means (e.g. nasal stent or special gum, pacifier, or other device, followed by GC/MS analysis or other interpretative technologies, as described by U.S. Pat. No. 9,480,461) for obtaining volatile/semi volatile compound spectra from samples. The consumer may be assigned a unique and anonymous identifier by means of their phone or otherwise. The breath profile may be loaded onto a device, then transmitted anonymously to a server. The breath profile is used to make a prediction, which may be made available to the consumer. Meanwhile the profile information is stored and associated with the anonymous consumer ID. If it is subsequently followed up by a formal medical diagnosis, the data becomes eligible for inclusion in the model, therefore improving subsequent predictions.

FIG. 4 shows an exemplary database for use in a system for predicting diseases. The system supports a number of disease indicators (inputs), focusing initially on those with a precedent for correlating volatile molecules with disease. Much of this information may be available in the form of publications in the scientific literature or data from clinical trials. This data is typically of high quality, but not always complete or abundant. It can be imported to the database and used to bootstrap the initial model building process, in order to provide prediction value while the data acquisition process is initiated. This process may be repeated with each new disease indication that is added to the system.

FIG. 5 is a block diagram illustrating an exemplary embodiment of a system for predicting diseases.

FIG. 6 is a block diagram illustrating an exemplary embodiment of a disease prediction server, such as is depicted by FIG. 5.

### DETAILED DESCRIPTION

The present disclosure generally related to systems and methods for predicting diseases. A system in accordance with some embodiments may accumulate information about the volatile, semi-volatile, and non-volatile organic compounds in breath/saliva. A goal may be to identify disease indications as early as possible, using non-invasive data collection via breath and alert consumers directly for follow-up with a health professional. The system that can make use of an ever growing collection of empirical evidence to make increasingly accurate predictions, at ever earlier stages, for a growing number of diseases that can be correlated with volatile, semi-volatile, and non-volatile emissions. This may be accomplished using a highly streamlined data collection process that is easy and inexpensive for patients and doctors, and techniques in machine learning based on contemporary approaches for solving big data problems. Exemplary techniques for extracting volatile and non-volatile chemicals from patients are described in U.S. Pat. No. 9,480,461, entitled “Methods for Extracting Chemicals from Nasal Cavities and Breath” and issued on Nov. 1, 2016, which is incorporated herein by reference.

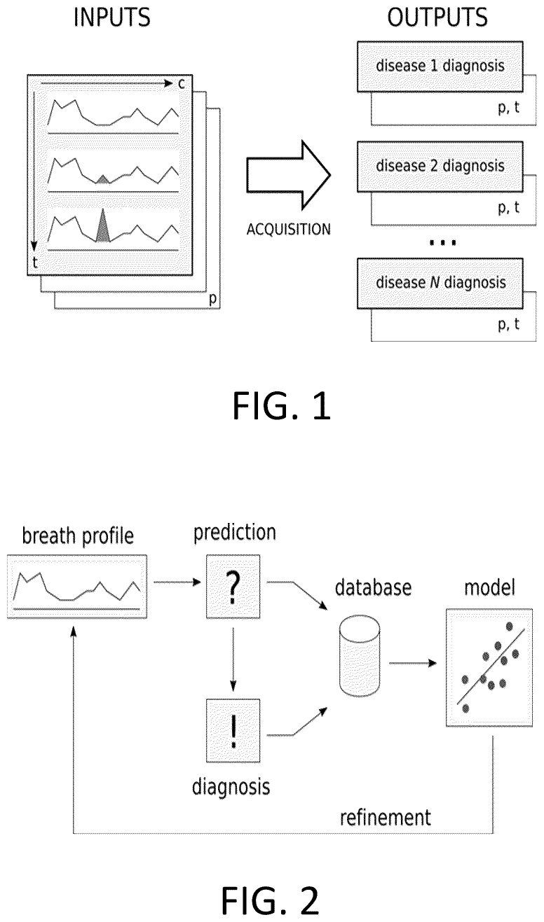

A readout that is collected from breath samples of consumers may be a spectrum of compounds and/or concentrations that are derived from (gc and lc) mass spectroscopy database and/or olfactory data integration for compound identification, cross referenced to a growing curated list of known compounds. These readouts can be taken at multiple times for any consumer over the course of years, and for multiple consumers. This represents one type of input data to the system. For each of these consumers, the system may accumulate or “predict” diagnosis events that correspond to diseases/biomarkers of that consumer and also apply learning collectively from other consumers to generate triggered indications as the system continues to learn, and these constitute output data (FIG. 1).

The successful assembly of a collection (e.g., database) of inputs and outputs defines information that may be used by machine learning techniques to provide disease predictions. Machine learning techniques may be used to identify profile patterns of compounds that are indicative of early indicators of a future disease. The system may make use of deep neural networks, with training/testing set partitioning to verify predictive ability. By including multiple timestamped measurements across the patient database, the system may be able to determine the maximum extent of its detection capability, i.e. how far back in time it is able to reach with acceptable predictivity.

An important characteristic of machine learning techniques, such as deep neural networks, is that they are able to identify patterns that are not only counterintuitive, but could not be determined without having access to a large amount of computing power and recent advances in deep learning algorithms. While some relatively straightforward patterns could be determined by expert technicians, the potential level of sensitivity that becomes possible with a large amount of high quality data and computing power represents a difference in kind compared to what is possible with analog data processing methods.

The ability to find counterintuitive patterns for correlating compound spectra with disease indicators can also be extended by augmenting the input conditions with other patient metadata (e.g. simple observables such as age, gender, smoking, diet, or even genetic markers). Clustering based on these additional conditions may improve the ability to subtarget pattern-to-disease correlation. The use of machine learning algorithms allows the possibility of establishing correlations that are counterintuitive and multidimensional, and are not plausible by traditional methods.

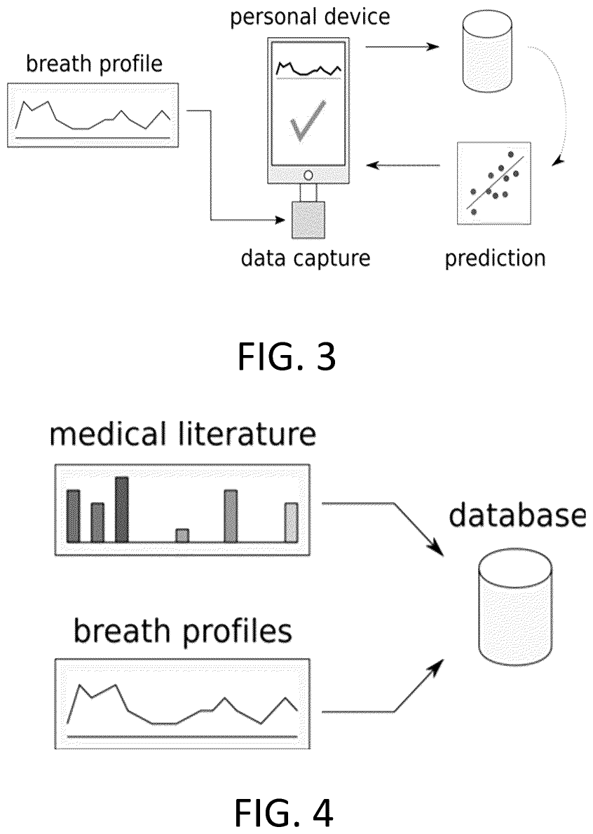

In one embodiment, a continuous data acquisition process is used to acquire data used by the system as shown by FIGS. 2 and 3. As an example, methods for gathering breath sample data from consumers described by U.S. Pat. No. 9,480,461, combined with the system's ability to link diagnosis events with doctors (another input), allow the system to accumulate an ever growing set of data that can be split into training/testing sets for model building, on a near real-time basis. The direct connection that the system has with the data gathering process addresses many of the applicability and reproducibility concerns that negatively affect other biomedical modeling exercises. Models may be rebuilt regularly as new data becomes available. One process involves iteratively improving the system's models with increases in data quantity, which forms a virtuous cycle: improved prediction means more successful early diagnoses, which further increases the data quality.

Another input may be aroma (olfactory) and the compound(s) that create the aroma that are aligned with different disease signatures. Using aroma allows for earlier recognition of disease due to aroma often being perceivable prior to compound detection utilizing existing technologies. Inputs can come from the same sources such as research, consumer reporting directly or through social media platforms and others.

In some embodiments, the system is designed to handle multiple disease indications, each of which has its own category of models for making predictions (and can also be used as input metadata, to help subcategorize). As new diseases are added, the system may be pre-populated with data from available sources, such as the medical literature and clinical trials as shown by FIG. 4. Transforming this data into the same form as is used for our own field collection method may involve curation.

One of the benefits of having a continuously learning system that improves the quality of disease models (as well as adding new disease models) is that it becomes possible to re-analyze historical consumer data. When consumers are found to be at risk for an improved or new disease indication, based on previously acquired data, the system may trigger an alert. The consumer may be contacted directly, with a suggestion that they seek medical diagnosis. Use of personal devices (such as phones) may be used to deliver these notifications.

All of the dimensions of the system may be designed to grow over time: as well as the number of disease indications and the volume of patient data, the list of volatile marker compounds may also grow as more relevant chemical structures are discovered. These may be integrated into the profiles, and tagged retroactively from the GC/MS data that corresponds to each of the breath profile datasets.

Gathering the data and storing it in compliance with all regulations regarding anonymity of medical information is a significant challenge: mapping of consumer identifiers with the breath data they generate, and the diagnoses that their doctors make, is a valuable part of the methodology.

The system may also include a financial tracking sub-system that allows for subscriptions payments for participation from users of the system, and it also may allow for integration direct back to users, if desired by system owner, to distribute a financial revenue share, based upon new learning and discoveries that traditionally had only been available to venture capitalists, investors, pharmaceutical companies and other like individuals/companies.

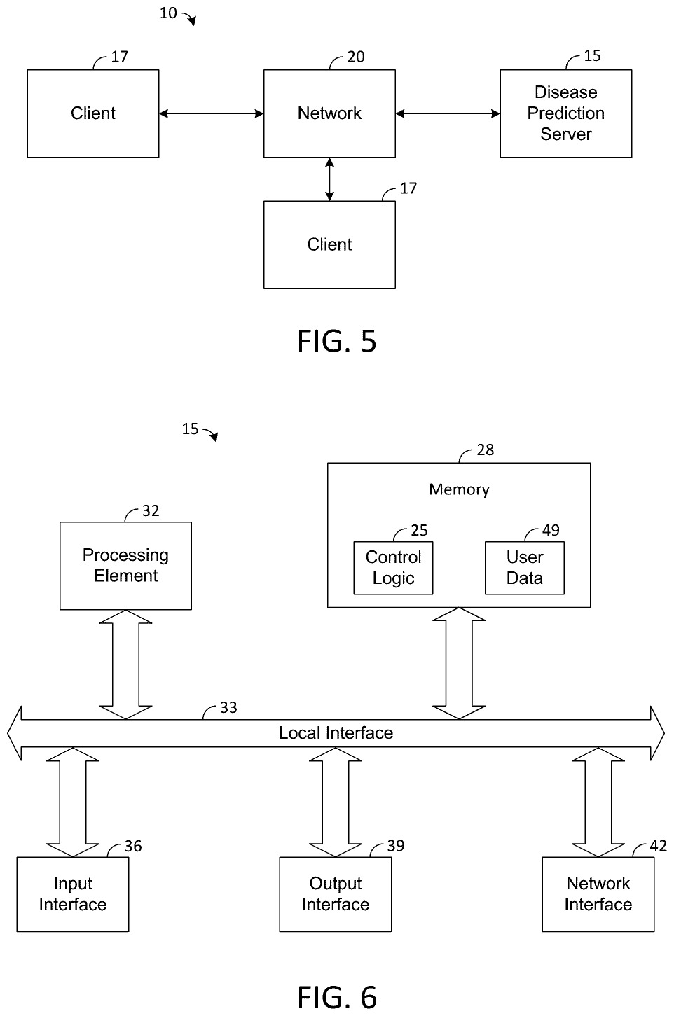

FIG. 5 depicts an exemplary embodiment of a system 10 for predicting diseases. In the embodiment depicted by FIG. 5, the system 10 comprises a disease prediction server 15 that is configured to predict diseases based on user information (e.g., information extracted from patients or other users) received from one or more clients 17. In this regard, the server 15 is communicatively coupled to the clients 17 through at least one network 20, such as a local area network (LAN), wide area network (WAN), or other type of network. As an example, the network 20 may comprise the Internet, and the server 15 may communicate with the clients 17 using transmission control protocol/Internet protocol (TCP/IP) or other type of protocol compatible with the network 20.

Each client 17 comprises a computing system having one or more communication devices for communicating with the server 15 through the network 20. As an example, a client 17 may comprise a desktop, laptop, or mainframe computer (or some other type of computer) having a modem or other type of device (e.g., a cellular or radio frequency (RF) radio) for communicating with the network 20. In another example, a client may comprise a cellular telephone (e.g., a smartphone) for processing and communicating data. Yet other types of devices may be used to implement any client 17.

The disease predicting server 15 may comprise any type of computing device, such as one more laptop, desktop, or mainframe computers, for performing the computing functions described herein. An exemplary embodiment of the server 15 is depicted by FIG. 6. As shown by FIG. 6, the server 15 comprises control logic 25 for controlling the functionality of the server 15 as will be described in more detail below. The control logic 25 can be implemented in software, hardware, firmware or any combination thereof. In the exemplary server 15 illustrated by FIG. 6, the control logic 25 is implemented in software and stored in memory 28 of the server 15.

Note that the control logic 25, when implemented in software, can be stored and transported on any computer-readable medium for use by or in connection with an instruction execution apparatus that can fetch and execute instructions. In the context of this document, a “computer-readable medium” can be any means that can contain or store a computer program for use by or in connection with an instruction execution apparatus.

The exemplary server 15 depicted by FIG. 6 comprises at least one conventional processor 32, such as a digital signal processor (DSP) or a central processing unit (CPU), that communicates to and drives the other elements within the server 15 via a local interface 33, which can include at least one bus. Furthermore, an input interface 36, for example, a keyboard or a mouse, can be used to input data from a user of the server 15, and an output interface 39, for example, a printer, monitor, liquid crystal display (LCD), or other display apparatus, can be used to output data to the user. Further, a network interface 42, such as at least one modem, cellular or RF radio, or other type of communication device, may be used to exchange data with the network 20 (FIG. 5).

During operation, the server 15 is configured to receive user information from one or more clients 17 (FIG. 5), and the control logic 25 is configured to store such information in memory 28 as user data 49. As an example, the user data 49 may define samples of chemicals (e.g., volatile or semi-volatile compounds) extracted from the breath of a patient or other user, and this information may be used to predict whether the patient or other user is likely to be afflicted with a specific disease in the future.

Note that there are various techniques that may be used to define the user data 49 for use by the server 15. In some embodiments, each user registers with the server 15 and is assigned or otherwise associated with a unique identifier during registration. If desired, the patient or other user (e.g., the patient's doctor) may use a client 17 to communicate and register the patient or other user with the server 15. In order to protect user confidentiality, the unique identifier may be anonymous so that the identity of the user cannot be ascertained by only analyzing the user data 49. That is, information that may be used to ascertain the identity of the user may not be provided to the server 15, though it is possible for such information to be provided in other embodiments. For each user, the user data 49 also defines contact information that can be used to contact the user or others affiliated with the user, such as the user's doctor or other healthcare professional, if a particular disease is predicted for the user. In some cases, the user identifier may also constitute the user contact information. As an example, a cellular telephone number may be used to both identify the user and contact the user if a disease is predicted.

Once a user is registered, user information may be submitted to the server 15, which then correlates such information with user's identifier in memory 28 (e.g., in a database). In some embodiments, a device having an absorbent material for absorbing or adsorbing chemicals (e.g., volatile or semi-volatile compounds) from the breaths of the user may be inserted in to the oral or nasal cavity of the user. In some cases, the device remains in the user's cavity for an extended time, such as several hours, so that even trace levels of chemicals in the user's breath are absorbed or adsorbed into the absorbent material in sufficient quantities so that the chemicals can be detected using conventional techniques for extracting and measuring chemicals from the absorbent material, such as gas chromatography. Exemplary techniques and devices for capturing and extracting chemicals from the breaths of the user are described by U.S. Pat. No. 9,480,461, which is incorporated herein by reference. Data indicative of the detected chemicals, including the measured amount of each detected chemical for a given sample, may be transmitted by a client 17 to the server 15. The message carrying such data, referred to herein as “sample data,” may include the user identifier used by the server 15 to associate the sample data with the user.

Over time, the process described above may be used to define several samples such that the user data 49 stored at the server 15 defines a history of samples for the user. As an example, the process described above may be performed annually (e.g., at each annual checkup for a patient) or some other regular or irregular period. Further, the process described above may be performed for a large number of users so that the user data 49 defines a history of samples for a large number of users, and this data may be used to not only predict diseases for the users but also to train the server 15 to better predict diseases, as will be described in more detail below.

In this regard, the control logic 25 may be configured to implement a machine learning algorithm to learn patterns in the samples for users indicative of disease and to use such patterns to predict when a user is likely to be afflicted with a particular disease in the future even if the user is not showing or knowingly experiencing any symptoms of the disease. In this regard, the control logic 25 may be trained using known training techniques for machine learning algorithms to learn the markers of certain diseases. As an example, a set of training data may be provided as input to a machine learning algorithm. Such training data may include the samples of users (where each sample indicates the chemicals extracted from breaths of the user) known to have been diagnosed with a particular disease. The training data may also include the samples of users who have not been diagnosed with diseases. The training data may also include the desired result (e.g., the diagnosed disease, if any) for each user so that the control logic 25 can learn how to map samples from a user to an accurate prediction of a disease, if any, consistent with the training data. That is, the control logic 25 uses machine learning in order to learn patterns in samples indicative of disease and uses these patterns as predictive markers for predicting when users are likely to be afflicted with certain diseases in the future.

In addition, once the server 28 begins operation and starts to process user data 49 for predicting diseases, the user data 49 acquired and used by the control logic 25 for performing disease prediction may also be used to continually train the control logic 25 in order to improve the results provided by the server 15 over time. As an example, once a doctor diagnoses a user with a disease, the doctor or other user may inform the server 15 of the diagnosis so that the control logic 25 can update the user's information in the user data 49 to indicate that the user has been so diagnosed. As an example, a doctor or other user may transmit information indicative of the diagnosis along with the user's identifier to the server 15, and the control logic 25 may use the user identifier to correlate the diagnosis with the set of user data 49 pertaining to such user in memory 28. Thus, over time, the control logic 25 may use the user data 49 as further training data to refine and update its mappings or algorithm to account for the diagnosis of users being monitored by the system 10.

In any event, as the server 15 receives samples for a given user, the control logic 25 stores the samples in memory 28 and analyzes the samples of the user to predict whether the user is likely to suffer from one or more diseases in the future. As an example, if the control logic 25 determines that user's samples satisfy a previously-learned marker for a particular disease, then the control logic 25 may predict that the user will be afflicted with the particular disease in the future. If such a prediction is made, the control logic 25 is configured to retrieve from the memory 28 the contact information that is associated with such user and use such contact information to send a notification to the user or others affiliated with the user (e.g., the user's doctor or other healthcare professional) to inform the user of the prediction. Such notification may identify the disease that has been predicted and a confidence level in the prediction. The user may then use such information to take one or more actions, such as beginning a treatment regime to treat the predicted disease, initiating one or more tests for diagnosing the predicted disease, or other action as may be appropriate.

If the user is confirmed to have the disease at some point, the server 15 may receive diagnosis information indicating a diagnosis confirming that the user has the disease. In response, the control logic 25 may update the user data 49 to indicate that the user has been diagnoses with the disease, and such information may be used to update the marker for the disease. As an example, once the user has been diagnosed with the disease, then the control logic 25 may use the samples associated with this user (along with sample from other users diagnosed to be afflicted with the same disease) learn the indicators that may be used to predict the disease according to the machine learning techniques described above. That is, as the server 15 is populated with more and more user data 49 the markers may be updated (e.g., re-learned) based on the user data 49, including new information added to the user data 49 over time. Further, if a user predicted to be afflicted with one disease is later diagnosed to have another disease, then the user's samples may be used to learn the marker for the later disease. Thus, the user data 49 used to predict some diseases may also be used to learn and update the markers for predicting other diseases. In addition, as markers are updated or new markers learned, the user data 49 may be reassessed based on these new markers. Thus, samples for a particular patient or other user that have been previously analyzed without a prediction of a particular disease may be analyzed again after a new (e.g., updated) marker for the disease is determined to reassess whether the user is predicted to be afflicted with the disease. Thus, the increased data collected by the system 10 is not only used to provide improved markers and predictions but also to continually reassess the samples that have been gathered so that the users associated with the samples can be evaluated based on new information obtained by the system 10.

Using machine learning techniques, as described above, the server 15 can learn patterns in even trace levels of chemicals extracted from breaths of users to predict diseases well before many such diseases could otherwise be detected using conventional techniques. Further, the techniques described herein are relatively low cost and can leverage a large amount of data to improve the results provided by the system.

Now, therefore, the following is claimed:

1. A disease prediction method, comprising:
receiving user data defining samples of chemicals extracted from breaths or saliva of a plurality of users over time, the samples including trace levels of the chemicals;
storing the user data in memory;
receiving diagnosis information indicative of diseases diagnosed for the users;
analyzing the user data and the diagnosis information with at least one processor according to a machine learning algorithm to learn a marker of a disease based at least on the trace levels of the chemicals, the marker corresponding to a pattern in the trace levels of the chemicals associated with ones of the plurality of users diagnosed with the disease;
determining with the at least one processor whether the marker is satisfied by a plurality of samples associated with a user, the plurality of samples defining a history of samples taken from the user over an extended time of at least one year, each of the plurality of samples associated with said user indicative of chemicals extracted from breaths or saliva of said user, wherein the determining comprises determining whether the plurality of samples associated with the user indicate the pattern;
predicting that said user will likely be afflicted with the disease if the marker is determined to be satisfied by the at least one processor; and
providing to at least one user a notice of the predicting by the at least one processor.

2. The method of claim 1, wherein the notice indicates a confidence of the at least one processor in the predicting.

3. The method of claim 1, wherein the user data further defines samples of aroma from the plurality of users, and wherein the predicting is based on the samples of aroma.

4. A disease prediction system, comprising:
memory for storing user data defining sets of samples for a plurality of users, each of the sets of the samples associated with a respective one of the plurality of users and indicative of chemicals extracted from breaths or saliva of the associated user over time, the sets of samples including trace levels of the chemicals extracted from the breaths or the saliva;
at least one processor programmed with instructions that when executed cause the at least one processor to:
receive diagnosis information indicative of diseases diagnosed for the plurality of users;
associate each of the diagnosed diseases with one of the plurality of users;
analyze the user data and the diagnosis information according to a machine learning algorithm to learn a first marker of a first disease based at least on the trace levels of the chemicals, the first marker corresponding to a pattern in the trace levels of the chemicals associated with ones of the plurality of users diagnosed with the first disease;
determine whether the first marker is satisfied by a set of the samples associated with a first user, each sample of the set of the samples associated with said first user indicative of chemicals extracted from breaths or saliva of said first user, wherein the set of the samples associated with said first user defines a history of samples taken from said first user over an extended time of at least one year, wherein the first marker is determined to be satisfied based on whether the set of the samples associated with said first user indicates the pattern;
predict that said first user will likely be afflicted with the first disease if the first marker is determined to be satisfied by the set of the samples associated with said first user; and
provide to at least one user a notice that said first user will be afflicted with the first disease if the first marker is determined to be satisfied by the set of the samples associated with said first user.

5. The system of claim 4, wherein the diagnosis information indicates that the first user is diagnosed to have a second disease, and wherein the instructions when executed further cause the at least one processor to:
analyze the user data and the diagnosis information according to the machine learning algorithm to learn a second marker of a second disease, wherein the at least one processor learns the second marker based on at least the set of the samples associated with said first user;
determine whether the second marker is satisfied by a set of the samples associated with a second user, each sample of the set of the samples associated with said second user indicative of chemicals extracted from breaths or saliva of said second user;
predict that said second user will be afflicted with the second disease if the second marker is determined to be satisfied by the set of the samples associated with said second user; and
provide to at least one user a notice that said second user will be afflicted with the second disease if the second marker is determined to be satisfied by the set of the samples associated with said second user.

6. The system of claim 4, wherein the notice indicates a confidence of the at least one processor in the predicting that said first user will be afflicted with the first disease.

7. A disease prediction method, comprising:
receiving user data defining samples of chemicals extracted from breaths or saliva of a plurality of users over time, the samples including trace levels of the chemicals;
storing the user data in memory;
receiving diagnosis information indicative of diseases diagnosed for the users;
analyzing the user data and the diagnosis information with at least one processor according to a machine learning algorithm to learn patterns in the trace levels of the chemicals associated with ones of the plurality of users diagnosed with the disease;
determining with the at least one processor whether a plurality of samples associated with a user is consistent with the learned patterns, the plurality of samples defining a history of samples taken from the user over an extended time of at least one year, each of the plurality of samples associated with said user indicative of chemicals extracted from breaths or saliva of said user;
predicting that said user will likely be afflicted with the disease based on the determining; and
providing to at least one user a notice of the predicting by the at least one processor.

8. The method of claim 7, wherein the user data further defines samples of aroma from the plurality of users, and wherein the predicting is based on the samples of aroma.
